(19) United States
(12) Patent Application Publication
(10) Pub. No.: US 2026/0226044 A1
(43) Pub. Date: Aug. 6, 2026

(54) PREPARATION OF AN S1P RECEPTOR MODULATOR

(71) Applicant: Oppilan Pharma Limited, San Diego, CA (US)

(72) Inventors: Venkat BOLLU, San Diego, CA (US); James COLLINS, San Diego, CA (US); John NUSS, San Diego, CA (US); Shendong YUAN, San Diego, CA (US); Scott Alan WOLCKENHAUER, San Diego, CA (US)

(21) Appl. No.: 19/147,237

(22) PCT Filed: Jan. 12, 2024
(86) PCT No.: PCT/US2024/011465

§ 371 Date: Jul. 10, 2025

Related U.S. Application Data
(60) Provisional application No. 63/479,932, filed on Jan. 13, 2023.

(51) Int. Cl. C07D 471/04; C07D 211/76

(52) U.S. Cl. CPC C07D 471/04; C07D 211/76

(57) ABSTRACT

Described herein is the preparation of the SIP receptor modulator tamuzimod ((R)-5-(2,5-dichloro-4-(5-(8-chloro-6-(tri-fluoromethyl)imidazo[1,2-a]pyridin-2-yl)-1,2,4-oxadiazol-3-yl)phenoxy)piperidin-2-one) and chemical intermediates used in the synthetic process.

### CROSS-REFERENCE

The application claims benefit of U.S. Provisional Application No. 63/479,932, filed on Jan. 13, 2023, which is herein incorporated by reference in its entirety.

### BACKGROUND OF THE INVENTION

The sphingosine-1-phosphate (SiP) receptors are a class of G protein-coupled receptors that are targets of the lipid signaling molecule sphingosine-1-phosphate. Sphingosine-1-phosphate (SiP) is a bioactive sphingolipid that has been demonstrated to induce many cellular processes, including those that result in platelet aggregation, cell proliferation, cell morphology, tumor-cell invasion, endothelial cell chemotaxis and angiogenesis, cytoskeletal re-arrangements in many cell types to regulate immune cell trafficking, vascular homeostasis and cell communication in the central nervous system (CNS) and in peripheral organ systems. SiP can bind with members of the endothelial cell differentiation gene family (EDG receptors) of plasma membrane-localized G protein-coupled receptors. To date, five members of this family have been identified as SiP receptors in different cell types, S1P1 (EDG-1), S1P2 (EDG-5), S1P3 (EDG-3), S1P4 (EDG-6) and S1P5 (EDG-8). SiP receptor modulators are compounds which signal as agonists or antagonists at one or more SiP receptors. Since SiP mediates a wide variety of cellular responses, SiP receptor modulators are promising targets for a variety of therapeutic indications.

### SUMMARY OF THE INVENTION

Described herein are processes for the synthesis of an SiP receptor modulator compound, wherein the compound is (R)-5-(2,5-dichloro-4-(5-(8-chloro-6-(trifluoromethyl)imidazo[1,2-a]pyridin-2-yl)-1,2,4-oxadiazol-3-yl)phenoxy)piperidin-2-one (Compound 1), or a pharmaceutically acceptable salt thereof.

In one aspect is a process for the preparation of (R)-5-(2,5-dichloro-4-(5-(8-chloro-6-(trifluoromethyl)imidazo[1,2-a]pyridin-2-yl)-1,2,4-oxadiazol-3-yl)phenoxy)piperidin-2-one (Compound 1):
A) the reaction of the compounds with the structures:

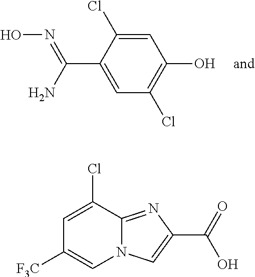 [SMILES: N/C(=N\O)c1cc(Cl)c(O)cc1Cl ; O=C(O)c1cn2cc(C(F)(F)F)cc(Cl)c2n1]

with HOBT and EDCI to produce a compound with the structure:

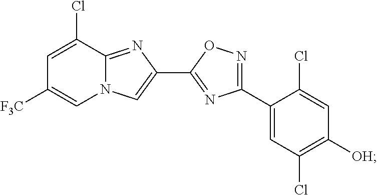 [SMILES: Oc1cc(Cl)c(-c2noc(-c3cn4cc(C(F)(F)F)cc(Cl)c4n3)n2)cc1Cl]

B) followed by the reaction of the compounds with the structures:

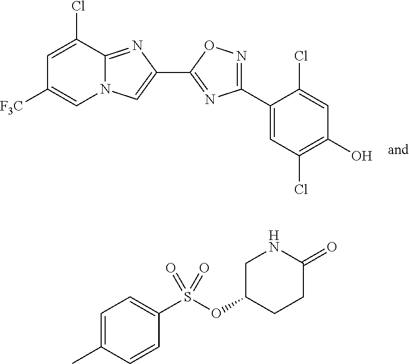 [SMILES: Oc1cc(Cl)c(-c2noc(-c3cn4cc(C(F)(F)F)cc(Cl)c4n3)n2)cc1Cl ; Cc1ccc(S(=O)(=O)O[C@H]2CCC(=O)NC2)cc1]

with cesium carbonate to produce (R)-5-(2,5-dichloro-4-(5-(8-chloro-6-(trifluoromethyl)imidazo[1,2-a]pyridin-2-yl)-1,2,4-oxadiazol-3-yl)phenoxy)piperidin-2-one (Compound 1) having the structure:

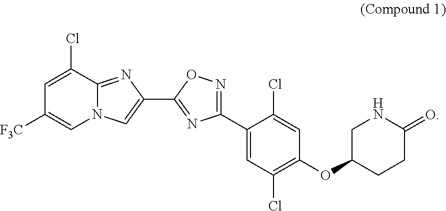 [SMILES: O=C1CC[C@@H](Oc2cc(Cl)c(-c3noc(-c4cn5cc(C(F)(F)F)cc(Cl)c5n4)n3)cc2Cl)CN1]

In another aspect is a process for the preparation of (R)-5-(2,5-dichloro-4-(5-(8-chloro-6-(trifluoromethyl)imidazo[1,2-a]pyridin-2-yl)-1,2,4-oxadiazol-3-yl)phenoxy)piperidin-2-one (Compound 1):
A) the reaction of the compounds with the structures:

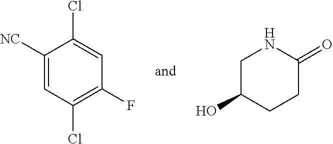 [SMILES: N#Cc1cc(Cl)c(F)cc1Cl ; O=C1CC[C@@H](O)CN1]

with potassium carbonate to produce a compound with the structure:

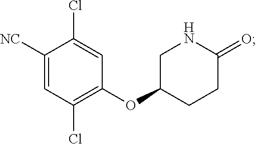 [SMILES: N#Cc1cc(Cl)c(O[C@@H]2CCC(=O)NC2)cc1Cl]

B) followed by the reaction of the compound with the structure:

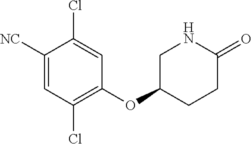 [SMILES: N#Cc1cc(Cl)c(O[C@@H]2CCC(=O)NC2)cc1Cl]

with hydroxylamine to produce a compound with the structure:

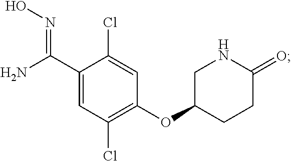 [SMILES: N/C(=N\O)c1cc(Cl)c(O[C@@H]2CCC(=O)NC2)cc1Cl]

C) the reaction of the compounds with the structures:

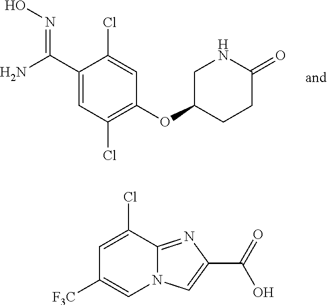

with HATU and DIEA to produce (R)-5-(2,5-dichloro-4-(5-(8-chloro-6-(trifluoromethyl)imidazo[1,2-a]pyridin-2-yl)-1,2,4-oxadiazol-3-yl)phenoxy)piperidin-2-one (Compound 1) having the structure:

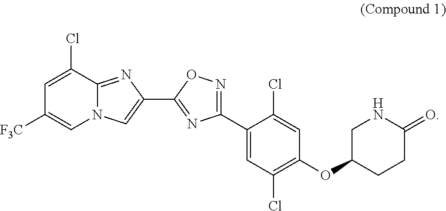 [SMILES: O=C1CC[C@@H](Oc2cc(Cl)c(-c3noc(-c4cn5cc(C(F)(F)F)cc(Cl)c5n4)n3)cc2Cl)CN1]

In some embodiments, the process for the preparation of (R)-5-(2,5-dichloro-4-(5-(8-chloro-6-(trifluoromethyl)imidazo[1,2-a]pyridin-2-yl)-1,2,4-oxadiazol-3-yl)phenoxy)piperidin-2-one (Compound 1) further comprises the reaction of (R)-5-(2,5-dichloro-4-(5-(8-chloro-6-(trifluoromethyl)imidazo[1,2-a]pyridin-2-yl)-1,2,4-oxadiazol-3-yl)phenoxy)piperidin-2-one (Compound 1) with acetic acid to produce (R)-5-(2,5-dichloro-4-(5-(8-chloro-6-(trifluoromethyl)imidazo[1,2-a]pyridin-2-yl)-1,2,4-oxadiazol-3-yl)phenoxy)piperidin-2-one acetic acid salt (Compound 1A).

In another aspect is a process for the preparation of (R)-5-(2,5-dichloro-4-(5-(8-chloro-6-(trifluoromethyl)imidazo[1,2-a]pyridin-2-yl)-1,2,4-oxadiazol-3-yl)phenoxy)piperidin-2-one (Compound 1):

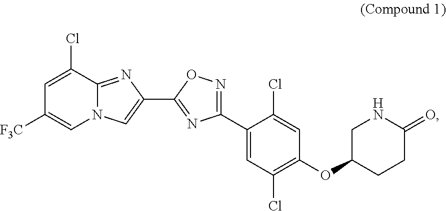 [SMILES: O=C1CC[C@@H](Oc2cc(Cl)c(-c3noc(-c4cn5cc(C(F)(F)F)cc(Cl)c5n4)n3)cc2Cl)CN1]

comprising contacting the compounds with the structures:

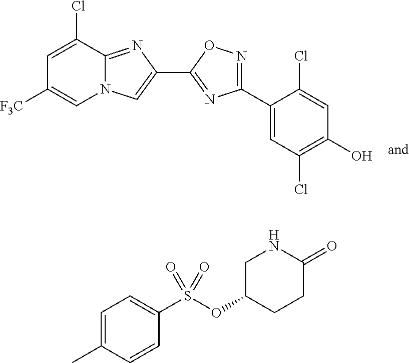 [SMILES: Oc1cc(Cl)c(-c2noc(-c3cn4cc(C(F)(F)F)cc(Cl)c4n3)n2)cc1Cl ; Cc1ccc(S(=O)(=O)O[C@H]2CCC(=O)NC2)cc1]

with a base in the presence of a solvent. In some embodiments, the base is selected from cesium carbonate, potassium carbonate, potassium phosphate, DBU, sodium t-butoxide, potassium t-butoxide, LiHMDS, NaHMDS, KHMDS, and mixtures thereof. In some embodiments, the base is cesium carbonate. In some embodiments, the base is a mixture of cesium carbonate and potassium phosphate. In some embodiments, the solvent is selected from dimethylacetamide, cyclopentyl methyl ether, DMF, NMP, THF, MeTHF, acetonitrile, toluene, and mixtures thereof. In some embodiments, the solvent is a mixture of dimethylacetamide and cyclopentyl methyl ether.

In some embodiments of a process for the preparation of (R)-5-(2,5-dichloro-4-(5-(8-chloro-6-(trifluoromethyl)imidazo[1,2-a]pyridin-2-yl)-1,2,4-oxadiazol-3-yl)phenoxy)piperidin-2-one (Compound 1), the compound with the structure:

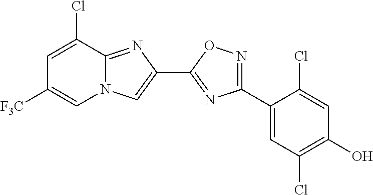 [SMILES: Oc1cc(Cl)c(-c2noc(-c3cn4cc(C(F)(F)F)cc(Cl)c4n3)n2)cc1Cl]

is prepared by a process comprising contacting the compounds with the structures:

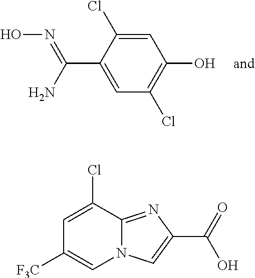 [SMILES: N/C(=N\O)c1cc(Cl)c(O)cc1Cl ; O=C(O)c1cn2cc(C(F)(F)F)cc(Cl)c2n1]

with a coupling agent in the presence of a solvent. In some embodiments, the coupling agent is selected from HOBT and EDCI, thionyl chloride, oxalyl chloride, COMU (1-[(1-(cyano-2-ethoxy-2-oxoethylideneaminooxy) dimethylaminomorpholino)]uronium hexafluorophosphate), HBTU (2-(1H-benzotriazole-1-yl)-1,1,3,3-tetramethyluroniumhexafluorophosphate), HCTU (O-(1H-6-chlorobenzotriazole-1-yl)-1,1,3,3-tetramethyluronium hexafluorophosphate), TCFH (chloro-N,N,N′,N′-tetramethylformamidinium hexafluorophosphate), phenylboronic acid, silicon tetrachloride, and tetraethyl orthosilicate. In some embodiments, the coupling agent is HOBT and EDCI. In some embodiments, the solvent is selected from N-methyl-2-pyrrolidone, DMF, DMAc, toluene, MeTHF, and acetonitrile. In some embodiments, the solvent is N-methyl-2-pyrrolidone.

In some embodiments of a process for the preparation of (R)-5-(2,5-dichloro-4-(5-(8-chloro-6-(trifluoromethyl)imidazo[1,2-a]pyridin-2-yl)-1,2,4-oxadiazol-3-yl)phenoxy)piperidin-2-one (Compound 1), the compound with the structure:

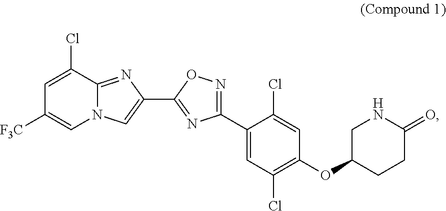 [SMILES: O=C1CC[C@@H](Oc2cc(Cl)c(-c3noc(-c4cn5cc(C(F)(F)F)cc(Cl)c5n4)n3)cc2Cl)CN1]

comprising contacting the compounds with the structures:

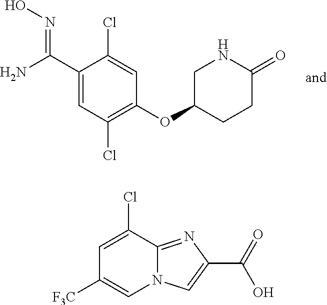

with a coupling agent and a base in the presence of a solvent. In some embodiments, the coupling agent is selected from HATU, thionyl chloride, oxalyl chloride, COMU (1-[(1-(cyano-2-ethoxy-2-oxoethylideneaminooxy) dimethylaminomorpholino)]uronium hexafluorophosphate), HBTU (2-(1H-benzotriazole-1-yl)-1,1,3,3-tetramethyluroniumhexafluorophosphate), HCTU (O-(1H-6-chlorobenzotriazole-1-yl)-1,1,3,3-tetramethyluronium hexafluorophosphate), TCFH (chloro-N,N,N′,N′-tetramethylformamidinium hexafluorophosphate), phenylboronic acid, silicon tetrachloride, and tetraethyl orthosilicate. In some embodiments, the coupling agent is HATU. In some embodiments, the base is selected from diisopropylethylamine, triethylamine, N-methylmorpholine, N-methylimidazole, and N-methylpiperidine. In some embodiments, the base is diisopropylethylamine. In some embodiments, the solvent is selected from dichloromethane, THF, MeTHF, acetonitrile, NMP, DMAc, DMF, and toluene. In some embodiments, the solvent is dichloromethane.

In some embodiments of a process for the preparation of (R)-5-(2,5-dichloro-4-(5-(8-chloro-6-(trifluoromethyl)imidazo[1,2-a]pyridin-2-yl)-1,2,4-oxadiazol-3-yl)phenoxy)piperidin-2-one (Compound 1), the compound with the structure:

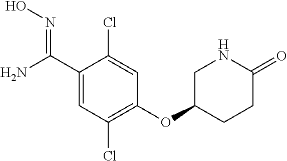 [SMILES: N/C(=N\O)c1cc(Cl)c(O[C@@H]2CCC(=O)NC2)cc1Cl]

is prepared by a process comprising contacting the compound with the structure:

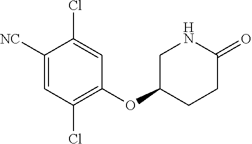 [SMILES: N#Cc1cc(Cl)c(O[C@@H]2CCC(=O)NC2)cc1Cl]

with hydroxylamine in the presence of a solvent. In some embodiments, the solvent is selected from dimethyl sulfoxide, DMF, DMAc, NMP, MeOH, EtOH, and 2-PrOH. In some embodiments, the solvent is dimethyl sulfoxide.

In some embodiments of a process for the preparation of (R)-5-(2,5-dichloro-4-(5-(8-chloro-6-(trifluoromethyl)imidazo[1,2-a]pyridin-2-yl)-1,2,4-oxadiazol-3-yl)phenoxy)piperidin-2-one (Compound 1), the compound with the structure:

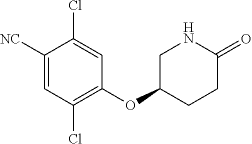 [SMILES: N#Cc1cc(Cl)c(O[C@@H]2CCC(=O)NC2)cc1Cl]

is prepared by a process comprising contacting the compounds with the structures:

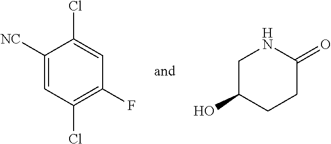 [SMILES: N#Cc1cc(Cl)c(F)cc1Cl ; O=C1CC[C@@H](O)CN1]

with a base in the presence of a solvent. In some embodiments, the base is selected from potassium carbonate, DBU, sodium t-butoxide, potassium t-butoxide, LiHMDS, NaHMDS, and KHMDS. In some embodiments, the base is potassium carbonate. In some embodiments, the solvent is selected from 2-butanone, DMF, DMAc, NMP, THF, and acetonitrile. In some embodiments, the solvent is 2-butanone.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

Good manufacturing practices are usually required for large scale manufacture of clinically useful drug candidates. Provided herein are certain processes and methods for the manufacture of(R)-5-(2,5-dichloro-4-(5-(8-chloro-6-(trifluoromethyl)imidazo[1,2-a]pyridin-2-yl)-1,2,4-oxadiazol-3-yl)phenoxy)piperidin-2-one (Compound 1), or a pharmaceutically acceptable salt or co-crystal thereof.

### Definitions

As used in the specification and appended claims, unless specified to the contrary, the following terms have the meaning indicated below.

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof.

When ranges are used herein for physical properties, such as molecular weight, or chemical properties, such as chemical formulae, all combinations and subcombinations of ranges and specific embodiments therein are intended to be included.

The term “about” when referring to a number or a numerical range means that the number or numerical range referred to is an approximation within experimental variability (or within statistical experimental error), and thus the number or numerical range varies between 1% and 15% of the stated number or numerical range.

The term “comprising” (and related terms such as “comprise” or “comprises” or “having” or “including”) is not intended to exclude that which in other certain embodiments, for example, an embodiment of any composition of matter, composition, method, or process, or the like, described herein, “consist of” or “consist essentially of” the described features.

The term “subject” or “patient” encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

As used herein, “treatment” or “treating” or “palliating” or “ameliorating” are used interchangeably herein. These terms refers to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are administered to a patient at risk of developing a particular disease, or to a patient reporting one or more of the physiological symptoms of a disease, even though a diagnosis of this disease has been made.

“Pharmaceutically acceptable salt” includes both acid and base addition salts. A pharmaceutically acceptable salt of any one of the compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, adipic acid, trifluoroacetic acid, propionic acid, glycolic acid, pywvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, adipates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S.M et al., “Pharmaceutical Salts,” Journal of Pharmaceutical Science, 66:1-19 (1997)). Acid addition salts of basic compounds are prepared by contacting the free base forms with a sufficient amount of the desired acid to produce the salt.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. In some embodiments, pharmaceutically acceptable base addition salts are formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

The term “pharmaceutical combination” as used herein, means a product that results from the mixing or combining of more than one active ingredient and includes both fixed and non-fixed combinations of the active ingredients. The term “fixed combination” means that the active ingredients, are both administered to a patient simultaneously in the form of a single entity or dosage. The term “non-fixed combination” means that the active ingredients, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The terms “co-administration” or the like, as used herein, are meant to encompass administration of the selected therapeutic agents to a single patient, and are intended to include treatment regimens in which the agents are administered by the same or different route of administration or at the same or different time.

The term “activator” is used in this specification to denote any molecular species that results in activation of the indicated receptor, regardless of whether the species itself binds to the receptor or a metabolite of the species binds to the receptor when the species is administered topically. Thus, the activator can be a ligand of the receptor or it can be an activator that is metabolized to the ligand of the receptor, i.e., a metabolite that is formed in tissue and is the actual ligand.

The term “antagonist” as used herein, refers to a small-molecule agent that binds to a nuclear hormone receptor and subsequently decreases the agonist induced transcriptional activity of the nuclear hormone receptor.

The term “agonist” as used herein, refers to a small-molecule agent that binds to a nuclear hormone receptor and subsequently increases nuclear hormone receptor transcriptional activity in the absence of a known agonist.

The term “inverse agonist” as used herein, refers to a small-molecule agent that binds to a nuclear hormone receptor and subsequently decreases the basal level of nuclear hormone receptor transcriptional activity that is present in the absence of a known agonist.

The term “modulate,” as used herein, means to interact with a target protein either directly or indirectly so as to alter the activity of the target protein, including, by way of example only, to inhibit the activity of the target, or to limit or reduce the activity of the target.

As used herein, the term “modulator” refers to a compound that alters an activity of a target. For example, a modulator can cause an increase or decrease in the magnitude of a certain activity of a target compared to the magnitude of the activity in the absence of the modulator. In certain embodiments, a modulator is an inhibitor, which decreases the magnitude of one or more activities of a target. In certain embodiments, an inhibitor completely prevents one or more activities of a target.

### Compounds

In some embodiments, the SiP receptor modulator described herein is (R)-5-(2,5-dichloro-4-(5-(8-chloro-6-(trifluoromethyl)imidazo[1,2-a]pyridin-2-yl)-1,2,4-oxadiazol-3-yl)phenoxy)piperidin-2-one (Compound 1), or a pharmaceutically acceptable salt or co-crystal thereof. Compound 1 has the structure:

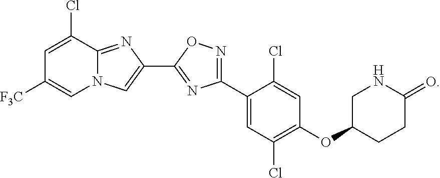 [SMILES: O=C1CC[C@@H](Oc2cc(Cl)c(-c3noc(-c4cn5cc(C(F)(F)F)cc(Cl)c5n4)n3)cc2Cl)CN1]

In some embodiments, the starting material for the synthesis of Compound 1 is

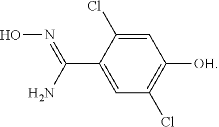 [SMILES: N/C(=N\O)c1cc(Cl)c(O)cc1Cl]

In some embodiments, an intermediate in the synthesis of Compound 1 is

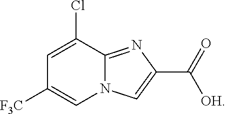 [SMILES: O=C(O)c1cn2cc(C(F)(F)F)cc(Cl)c2n1]

In some embodiments, an intermediate in the synthesis of Compound 1 is

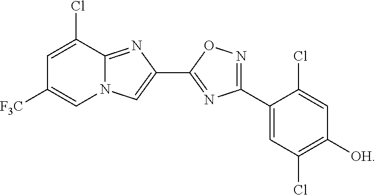 [SMILES: Oc1cc(Cl)c(-c2noc(-c3cn4cc(C(F)(F)F)cc(Cl)c4n3)n2)cc1Cl]

In some embodiments, an intermediate in the synthesis of Compound 1 is

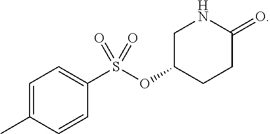 [SMILES: Cc1ccc(S(=O)(=O)O[C@H]2CCC(=O)NC2)cc1]

In some embodiments, a starting material in the synthesis of Compound 1 is

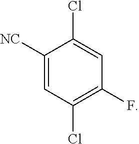 [SMILES: N#Cc1cc(Cl)c(F)cc1Cl]

In some embodiments, a starting material in the synthesis of Compound 1 is

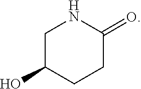

In some embodiments, a starting material in the synthesis of Compound 1 is

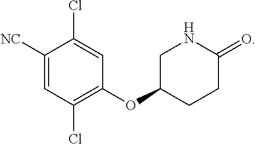 [SMILES: N#Cc1cc(Cl)c(O[C@@H]2CCC(=O)NC2)cc1Cl]

In some embodiments, an intermediate in the synthesis of Compound 1 is

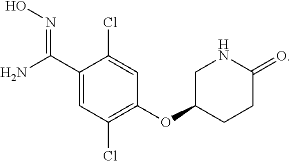 [SMILES: N/C(=N\O)c1cc(Cl)c(O[C@@H]2CCC(=O)NC2)cc1Cl]

### Further Forms of Compounds

The compounds described herein may in some cases exist as diastereomers, enantiomers, or other stereoisomeric forms. The compounds presented herein include all diastereomeric, enantiomeric, and epimeric forms as well as the appropriate mixtures thereof. Separation of stereoisomers may be performed by chromatography or by the forming diastereomeric and separation by recrystallization, or chromatography, or any combination thereof. (Jean Jacques, Andre Collet, Samuel H. Wilen, “Enantiomers, Racemates and Resolutions”, John Wiley And Sons, Inc., 1981, herein incorporated by reference for this disclosure). Stereoisomers may also be obtained by stereoselective synthesis.

In some situations, compounds may exist as tautomers. All tautomers are included within the formulas described herein.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds of the invention, or by separately reacting a purified compound in its free form with a suitable acid or base, and isolating the salt thus formed.

In some embodiments, the pharmaceutically acceptable salt of Compound 1 is an acetate, adipate, benzoate, besylate, bitartrate, carbonate, citrate, fumarate, gluconate, hydrobromide, hydrochloride, maleate, mesylate, nitrate, phosphate, salicylate, succinate, sulfate, or tartrate salt. In some embodiments, the pharmaceutically acceptable salt of Compound 1 is a mono-hydrochloride salt. In further embodiments, the pharmaceutically acceptable salt of Compound 1 is a mono-hydrochloride salt. In some embodiments, the pharmaceutically acceptable salt of Compound 1 is an adipic acid salt.

### Solvates

In some embodiments, the compounds described herein exist as solvates. The invention provides for methods of treating diseases by administering such solvates. The invention further provides for methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein are conveniently prepared or formed during the processes described herein. By way of example only, hydrates of the compounds described herein are conveniently prepared by recrystallization from an aqueous/organic solvent mixture, using organic solvents including, but not limited to, dioxane, tetrahydrofuran or methanol. In addition, the compounds provided herein exist in unsolvated as well as solvated forms. In general, the solvated forms are considered equivalent to the unsolvated forms for the purposes of the compounds and methods provided herein.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that are incorporated into compounds of the invention include isotopes of hydrogen, carbon, nitrogen, oxygen, phosphorous, sulfur, fluorine and chloride, such as ²H, ³H, ¹³C, ¹⁴C, ¹⁵N, ¹⁸O, ¹⁷O, ³¹P, ³²p, ³⁵S, ¹⁸F, and ³⁶Cl, respectively. Compounds described herein, and pharmaceutically acceptable salts, esters, solvate, hydrates or derivatives thereof which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i. e., ³H and carbon-14, i. e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements. Increased levels of deuterium incorporation produce a detectable kinetic isotope effect (KIE) that may affect the pharmacokinetic, pharmacologic and/or toxicologic parameters of Compound 1 in comparison to Compound 1 having naturally occurring levels of deuterium. In some embodiments, the isotopically labeled compound, or a pharmaceutically acceptable salt thereof, is prepared by any suitable method.

In some embodiments, the compounds described herein are labeled by other means, including, but not limited to, the use of chromophores or fluorescent moieties, bioluminescent labels, or chemiluminescent labels.

### Process for Preparation

In some embodiments, the synthesis of compounds described herein are accomplished using means described in the chemical literature, using the methods described herein, or by a combination thereof. In addition, solvents, temperatures and other reaction conditions presented herein may vary.

In other embodiments, the starting materials and reagents used for the synthesis of the compounds described herein are synthesized or are obtained from commercial sources, such as, but not limited to, Sigma-Aldrich, FischerScientific (Fischer Chemicals), and AcrosOrganics. In further embodiments, the compounds described herein, and other related compounds having different substituents are synthesized using techniques and materials described herein as well as those that are recognized in the field, such as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey and Sundberg, Advanced Organic Chemistry 4^{th} Ed., Vols. A and B (Plenum 2000, 2001), and Green and Wuts, Protective Groups in Organic Synthesis 3^{rd} Ed., (Wiley 1999) (all of which are incorporated by reference for such disclosure). General methods for the preparation of compounds as disclosed herein may be derived from reactions and the reactions may be modified by the use of appropriate reagents and conditions, for the introduction of the various moieties found in the formulae as provided herein.

In some embodiments is a process for the preparation of (R)-5-(2,5-dichloro-4-(5-(8-chloro-6-(trifluoromethyl)imidazo[1,2-a]pyridin-2-yl)-1,2,4-oxadiazol-3-yl)phenoxy)piperidin-2-one (Compound 1):
A) the reaction of the compounds with the structures:

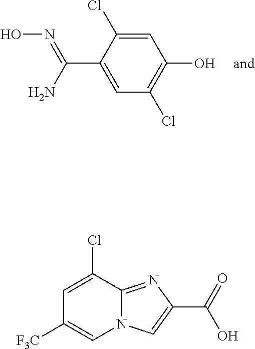 [SMILES: N/C(=N\O)c1cc(Cl)c(O)cc1Cl ; O=C(O)c1cn2cc(C(F)(F)F)cc(Cl)c2n1]

with HOBT and EDCI to produce a compound with the structure:

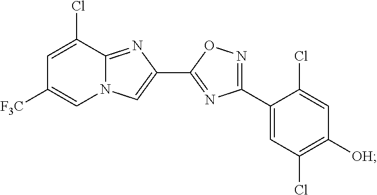 [SMILES: Oc1cc(Cl)c(-c2noc(-c3cn4cc(C(F)(F)F)cc(Cl)c4n3)n2)cc1Cl]

B) followed by the reaction of the compounds with the structures:

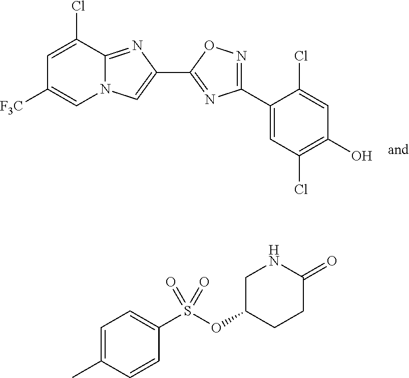 [SMILES: Oc1cc(Cl)c(-c2noc(-c3cn4cc(C(F)(F)F)cc(Cl)c4n3)n2)cc1Cl ; Cc1ccc(S(=O)(=O)O[C@H]2CCC(=O)NC2)cc1]

with cesium carbonate to produce (R)-5-(2,5-dichloro-4-(5-(8-chloro-6-(trifluoromethyl)imidazo[1,2-a]pyridin-2-yl)-1,2,4-oxadiazol-3-yl)phenoxy)piperidin-2-one (Compound 1) having the structure:

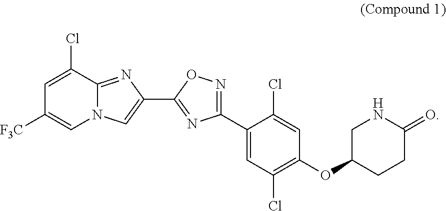 [SMILES: O=C1CC[C@@H](Oc2cc(Cl)c(-c3noc(-c4cn5cc(C(F)(F)F)cc(Cl)c5n4)n3)cc2Cl)CN1]

In some embodiments, the process further comprises the reaction of (R)-5-(2,5-dichloro-4-(5-(8-chloro-6-(trifluoromethyl)imidazo[1,2-a]pyridin-2-yl)-1,2,4-oxadiazol-3-yl)phenoxy)piperidin-2-one (Compound 1) with acetic acid to produce (R)-5-(2,5-dichloro-4-(5-(8-chloro-6-(trifluoromethyl)imidazo[1,2-a]pyridin-2-yl)-1,2,4-oxadiazol-3-yl)phenoxy)piperidin-2-one acetic acid salt (Compound 1A).

In some embodiments is a process for the preparation of (R)-5-(2,5-dichloro-4-(5-(8-chloro-6-(trifluoromethyl)imidazo[1,2-a]pyridin-2-yl)-1,2,4-oxadiazol-3-yl)phenoxy)piperidin-2-one (Compound 1):

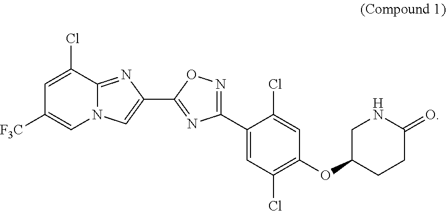 [SMILES: O=C1CC[C@@H](Oc2cc(Cl)c(-c3noc(-c4cn5cc(C(F)(F)F)cc(Cl)c5n4)n3)cc2Cl)CN1]

comprising contacting the compounds with the structures:

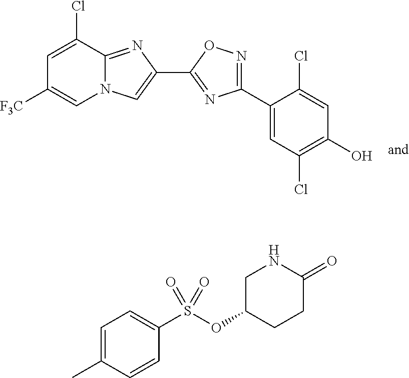 [SMILES: Oc1cc(Cl)c(-c2noc(-c3cn4cc(C(F)(F)F)cc(Cl)c4n3)n2)cc1Cl ; Cc1ccc(S(=O)(=O)O[C@H]2CCC(=O)NC2)cc1]

with a base in the presence of a solvent. In some embodiments, the base is selected from cesium carbonate, potassium carbonate, potassium phosphate, DBU, sodium t-butoxide, potassium t-butoxide, LiHMDS, NaHMDS, KHMDS, and mixtures thereof. In some embodiments, the base is cesium carbonate. In some embodiments, the base is potassium carbonate. In some embodiments, the base is potassium phosphate. In some embodiments, the base is DBU. In some embodiments, the base is sodium t-butoxide. In some embodiments, the base is potassium t-butoxide. In some embodiments, the base is LiHMDS. In some embodiments, the base is NaHMDS. In some embodiments, the base is KHMDS. In some embodiments, the base is a mixture of cesium carbonate and potassium phosphate. In some embodiments, the solvent is selected from dimethylacetamide, cyclopentyl methyl ether, DMF, NMP, THF, MeTHF, acetonitrile, toluene, and mixtures thereof. In some embodiments, the solvent is dimethylacetamide. In some embodiments, the solvent is cyclopentyl methyl ether. In some embodiments, the solvent is DMF. In some embodiments, the solvent is NMP. In some embodiments, the solvent is THF. In some embodiments, the solvent is MeTHF. In some embodiments, the solvent is acetonitrile. In some embodiments, the solvent is toluene. In some embodiments, the solvent is a mixture of dimethylacetamide and cyclopentyl methyl ether.

In some embodiments of a process for the preparation of (R)-5-(2,5-dichloro-4-(5-(8-chloro-6-(trifluoromethyl)imidazo[1,2-a]pyridin-2-yl)-1,2,4-oxadiazol-3-yl)phenoxy)piperidin-2-one (Compound 1), the compound with the structure:

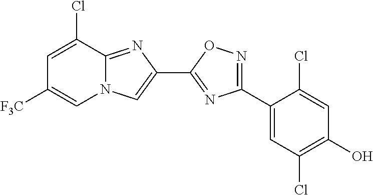 [SMILES: Oc1cc(Cl)c(-c2noc(-c3cn4cc(C(F)(F)F)cc(Cl)c4n3)n2)cc1Cl]

is prepared by a process comprising contacting the compounds with the structures:

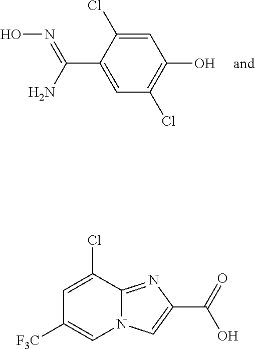 [SMILES: N/C(=N\O)c1cc(Cl)c(O)cc1Cl ; O=C(O)c1cn2cc(C(F)(F)F)cc(Cl)c2n1]

with a coupling agent in the presence of a solvent. In some embodiments, the coupling agent is selected from HOBT and EDCI, thionyl chloride, oxalyl chloride, COMU (1-[(1-(cyano-2-ethoxy-2-oxoethylideneaminooxy) dimethylaminomorpholino)]uronium hexafluorophosphate), HBTU (2-(1H-benzotriazole-1-yl)-1,1,3,3-tetramethyluroniumhexafluorophosphate), HCTU (O-(1H-6-chlorobenzotriazole-1-yl)-1,1,3,3-tetramethyluronium hexafluorophosphate), TCFH (chloro-N,N,N′,N′-tetramethylformamidinium hexafluorophosphate), phenylboronic acid, silicon tetrachloride, and tetraethyl orthosilicate. In some embodiments, the coupling agent is HOBT and EDCI. In some embodiments, the coupling agent is thionyl chloride. In some embodiments, the coupling agent is oxalyl chloride. In some embodiments, the coupling agent is COMU (1-[(1-(cyano-2-ethoxy-2-oxoethylideneaminooxy) dimethylaminomorpholino)]uronium hexafluorophosphate). In some embodiments, the coupling agent is HBTU (2-(1H-benzotriazole-1-yl)-1,1,3,3-tetramethyluronium hexafluorophosphate). In some embodiments, the coupling agent is HCTU (O-(1H-6-chlorobenzotriazole-1-yl)-1,1,3,3-tetramethyluronium hexafluorophosphate). In some embodiments, the coupling agent is TCFH (chloro-N,N,N′,N′-tetramethylformamidinium hexafluorophosphate). In some embodiments, the coupling agent is phenylboronic acid. In some embodiments, the coupling agent is silicon tetrachloride. In some embodiments, the coupling agent is tetraethyl orthosilicate. In some embodiments, the solvent is selected from N-methyl-2-pyrrolidone, DMF, DMAc, toluene, MeTHF, and acetonitrile. In some embodiments, the solvent is N-methyl-2-pyrrolidone. In some embodiments, the solvent is DMF. In some embodiments, the solvent is DMAc. In some embodiments, the solvent is toluene. In some embodiments, the solvent is MeTHF. In some embodiments, the solvent is acetonitrile.

In some embodiments of a process for the preparation of (R)-5-(2,5-dichloro-4-(5-(8-chloro-6-(trifluoromethyl)imidazo[1,2-a]pyridin-2-yl)-1,2,4-oxadiazol-3-yl)phenoxy)piperidin-2-one (Compound 1), the compound with the structure:

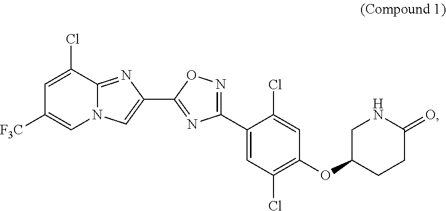 [SMILES: O=C1CC[C@@H](Oc2cc(Cl)c(-c3noc(-c4cn5cc(C(F)(F)F)cc(Cl)c5n4)n3)cc2Cl)CN1]

comprising contacting the compounds with the structures:

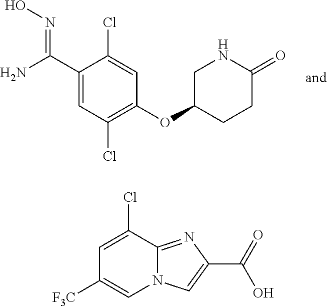

with a coupling agent and a base in the presence of a solvent. In some embodiments, the coupling agent is selected from HATU, thionyl chloride, oxalyl chloride, COMU (1-[(1-(cyano-2-ethoxy-2-oxoethylideneaminooxy) dimethylaminomorpholino)]uronium hexafluorophosphate), HBTU (2-(1H-benzotriazole-1-yl)-1,1,3,3-tetramethyluroniumhexafluorophosphate), HCTU (O-(1H-6-chlorobenzotriazole-1-yl)-1,1,3,3-tetramethyluronium hexafluorophosphate), TCFH (chloro-N,N,N′,N′-tetramethylformamidinium hexafluorophosphate), phenylboronic acid, silicon tetrachloride, and tetraethyl orthosilicate. In some embodiments, the coupling agent is HATU. In some embodiments, the coupling agent is thionyl chloride. In some embodiments, the coupling agent is oxalyl chloride. In some embodiments, the coupling agent is COMU (1-[(1-(cyano-2-ethoxy-2-oxoethylideneaminooxy) dimethylaminomorpholino)]uronium hexafluorophosphate). In some embodiments, the coupling agent is HBTU (2-(1H-benzotriazole-1-yl)-1,1,3,3-tetramethyluronium hexafluorophosphate). In some embodiments, the coupling agent is HCTU (O-(1H-6-chlorobenzotriazole-1-yl)-1,1,3,3-tetramethyluronium hexafluorophosphate). In some embodiments, the coupling agent is TCFH (chloro-N,N,N′,N′-tetramethylformamidinium hexafluorophosphate). In some embodiments, the coupling agent is phenylboronic acid. In some embodiments, the coupling agent is silicon tetrachloride. In some embodiments, the coupling agent is tetraethyl orthosilicate. In some embodiments, the base is selected from diisopropylethylamine, triethylamine, N-methylmorpholine, N-methylimidazole, and N-methylpiperidine. In some embodiments, the base is triethylamine. In some embodiments, the base is diisopropylethylamine. In some embodiments, the base is N-methylmorpholine. In some embodiments, the base is N-methylpyrrolidine. In some embodiments, the base is N-methylpiperidine. In some embodiments, the solvent is selected from dichloromethane, THF, MeTHF, acetonitrile, NMP, DMAc, DMF, and toluene. In some embodiments, the solvent is dichloromethane. In some embodiments, the solvent is THF. In some embodiments, the solvent is N-methylpyrrolidone (NMP). In some embodiments, the solvent is MeTHF. In some embodiments, the solvent is acetonitrile. In some embodiments, the solvent is DMAc. In some embodiments, the solvent is DMF. In some embodiments, the solvent is toluene.

In some embodiments of a process for the preparation of (R)-5-(2,5-dichloro-4-(5-(8-chloro-6-(trifluoromethyl)imidazo[1,2-a]pyridin-2-yl)-1,2,4-oxadiazol-3-yl)phenoxy)piperidin-2-one (Compound 1), the compound with the structure:

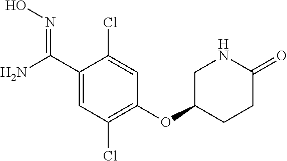 [SMILES: N/C(=N\O)c1cc(Cl)c(O[C@@H]2CCC(=O)NC2)cc1Cl]

is prepared by a process comprising contacting the compound with the structure:

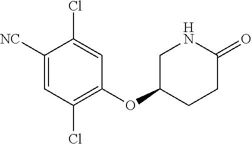 [SMILES: N#Cc1cc(Cl)c(O[C@@H]2CCC(=O)NC2)cc1Cl]

with hydroxylamine in the presence of a solvent. In some embodiments, the solvent is selected from dimethyl sulfoxide, DMF, DMAc, NMP, MeOH, EtOH, and 2-PrOH. In some embodiments, the solvent is dimethyl sulfoxide. In some embodiments, the solvent is DMF. In some embodiments, the solvent is DMAc. In some embodiments, the solvent is NMP. In some embodiments, the solvent is MeOH. In some embodiments, the solvent is EtOH. In some embodiments, the solvent is 2-PrOH.

In some embodiments of a process for the preparation of (R)-5-(2,5-dichloro-4-(5-(8-chloro-6-(trifluoromethyl)imidazo[1,2-a]pyridin-2-yl)-1,2,4-oxadiazol-3-yl)phenoxy)piperidin-2-one (Compound 1), the compound with the structure:

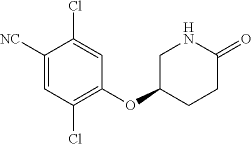 [SMILES: N#Cc1cc(Cl)c(O[C@@H]2CCC(=O)NC2)cc1Cl]

is prepared by a process comprising contacting the compounds with the structures:

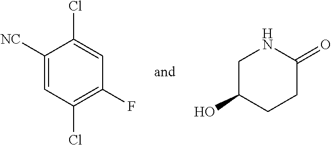 [SMILES: N#Cc1cc(Cl)c(F)cc1Cl ; O=C1CC[C@@H](O)CN1]

with a base in the presence of a solvent. In some embodiments, the base is selected from potassium carbonate, DBU, sodium t-butoxide, potassium t-butoxide, LiHMDS, NaHMDS, and KHMDS. In some embodiments, the base is potassium carbonate. In some embodiments, the base is potassium carbonate. In some embodiments, the base is DBU. In some embodiments, the base is triethylamine. In some embodiments, the base is sodium t-butoxide. In some embodiments, the base is LiHMDS. In some embodiments, the base is NaHMDS. In some embodiments, the base is KHMDS. In some embodiments, the solvent is selected from 2-butanone, DMF, DMAc, NMP, THF, and acetonitrile. In some embodiments, the solvent is 2-butanone. In some embodiments, the solvent is DMF. In some embodiments, the solvent is DMAc. In some embodiments, the solvent is NMP. In some embodiments, the solvent is THF. In some embodiments, the solvent is acetonitrile.

In some embodiments is a compound with the structure:

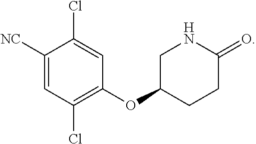 [SMILES: N#Cc1cc(Cl)c(O[C@@H]2CCC(=O)NC2)cc1Cl]

In some embodiments is a compound with the structure:

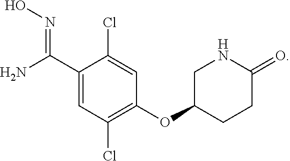 [SMILES: N/C(=N\O)c1cc(Cl)c(O[C@@H]2CCC(=O)NC2)cc1Cl]

### Pharmaceutical Compositions and Methods of Administration

Administration of Compound 1 described herein can be in any pharmacological form including a therapeutically effective amount of Compound 1 alone or in combination with a pharmaceutically acceptable carrier.

Pharmaceutical compositions may be formulated in a conventional manner using one or more physiologically acceptable carriers including excipients and auxiliaries which facilitate processing of the active compounds into preparations which can be used pharmaceutically. Proper formulation is dependent upon the route of administration chosen. Additional details about suitable excipients for pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkinsl999), herein incorporated by reference for such disclosure.

A pharmaceutical composition, as used herein, refers to a mixture of Compound 1 described herein, with other chemical components, such as carriers, stabilizers, diluents, dispersing agents, suspending agents, thickening agents, and/or excipients. The pharmaceutical composition facilitates administration of the compound to an organism. In practicing the methods of treatment or use provided herein, therapeutically effective amounts of compounds described herein are administered in a pharmaceutical composition to a mammal having a disease, disorder, or condition to be treated. In some embodiments, the mammal is a human. A therapeutically effective amount can vary widely depending on the severity of the disease, the age and relative health of the subject, the potency of the compound used and other factors. Compound 1 can be used singly or in combination with one or more therapeutic agents as components of mixtures (as in combination therapy).

The pharmaceutical formulations described herein can be administered to a subject by multiple administration routes, including but not limited to, oral, parenteral (e.g., intravenous, subcutaneous, intramuscular), intranasal, buccal, topical, rectal, or transdermal administration routes. Moreover, the pharmaceutical compositions described herein, which include Compound 1 described herein, can be formulated into any suitable dosage form, including but not limited to, aqueous oral dispersions, liquids, gels, syrups, elixirs, slurries, suspensions, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

In some embodiments, Compound 1 is formulated in a tablet dosage form. In some embodiments, Compound 1 is formulated in a capsule dosage form. In some embodiments, Compound 1 is formulated in a suspension dosage form. In some embodiments, Compound 1 is formulated as powder-in-capsule dosage form. In some embodiments, Compound 1 is formulated as a powder-in-bottle for reconstitution as a suspension.

Pharmaceutical compositions including a compound described herein may be manufactured in a conventional manner, such as, by way of example only, by means of conventional mixing, dissolving, granulating, dragee-making, levigating, emulsifying, encapsulating, entrapping or compression processes.

Dose administration can be repeated depending upon the pharmacokinetic parameters of the dosage formulation and the route of administration used.

It is especially advantageous to formulate compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms are dictated by and directly dependent on (a) the unique characteristics of Compound 1 and the particular therapeutic effect to be achieved and (b) the limitations inherent in the art of compounding such an active compound for the treatment of sensitivity in individuals. The specific dose can be readily calculated by one of ordinary skill in the art, e.g., according to the approximate body weight or body surface area of the patient or the volume of body space to be occupied. The dose will also be calculated dependent upon the particular route of administration selected. Further refinement of the calculations necessary to determine the appropriate dosage for treatment is routinely made by those of ordinary skill in the art. Exact dosages are determined in conjunction with standard dose-response studies. It will be understood that the amount of the composition actually administered will be determined by a practitioner, in the light of the relevant circumstances including the condition or conditions to be treated, the choice of composition to be administered, the age, weight, and response of the individual patient, the severity of the patient's symptoms, and the chosen route of administration.

### EXAMPLES

All chemicals, reagents, and solvents were purchased from commercial sources when available and used without further purification.

Standard abbreviations and acronyms as defined in J. Org. Chem. 2007 72(1): 23A-24A are used herein. Other abbreviations and acronyms used herein are as follows:

| | |
|---|---|
| ACN | acetonitrile |
| AcOH | acetic acid |
| BINAP | (2,2′-bis(diphenylphosphino)-1,1′-binaphthyl) |
| DCM | dichloromethane |
| DMF | dimethylformamide |
| DMP | Dess-Martin periodinane |
| DMSO | dimethyl sulfoxide |
| dppf | (diphenylphosphino)ferrocene |
| EtOAc | ethyl acetate |
| EtOH | ethanol |
| eq | equivalent |
| HBTU | N,N,N′,N′-tetramethyl-O-(1H- |
| | benzotriazol-1-yl)uronium hexafluorophosphate |
| LC-MS | liquid chromatography-mass spectrometry |
| MeOH | methanol |
| TEA | triethylamine |
| TFA | trifluoroacetic acid |
| RT | room temperature |

### Example 1: Synthesis of (R)-5-(2,5-dichloro-4-(5-(8-chloro-6-(trifluoromethyl)imidazo[1,2-a]pyridin-2-yl)-1,2,4-oxadiazol-3-yl)phenoxy)piperidin-2-one (Compound 1)

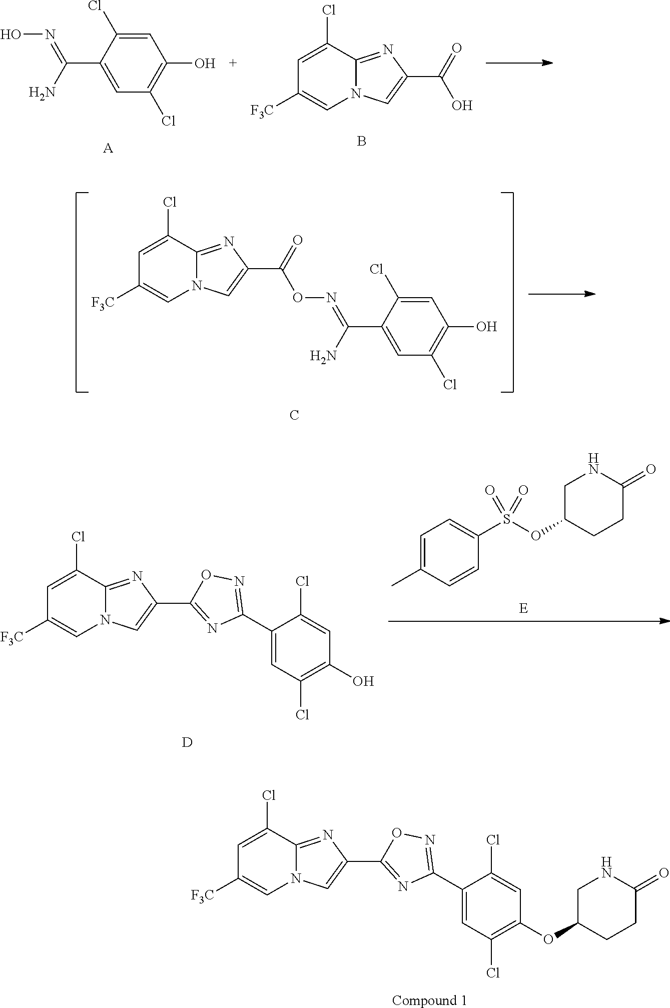

To a solution of compound B (62.8 g, 1.05 eq) in NMP (250.0 mL) at 10° C. was added HOBt (36.7 g, 1.2 eq). The mixture was stirred for 0.5 h at 10° C. and EDCI (52.0 g, 1.2 eq) was added. The mixture was stirred for 2 h at 10° C. and then compound A (50.0 g) was added. The reaction mixture was warmed to 25° C. and stirred for 2 h. The mixture was warmed to 80° C. and stirred for 7 h. The mixture was cooled to 50° C. and water (300 mL) was added. The mixture was stirred for 1 h. The mixture was cooled to 25° C. and stirred for 5 h. The mixture was filtered and the filter cake was washed with H₂O (2×100 mL). The wet cake with dissolved in 2-MeTHF (250 mL) and stirred for 1 h at 60° C. Heptane (500 mL) was added at 60° C. and the mixture was stirred for 1 h. The mixture was cooled to 25° C. and filtered. The filter cake was washed with n-heptane (2×100 mL) and dried for 24 h at 60° C. to afford compound D (88.9 g, 73%). ¹HNMR (400 MHz, DMSO) δ 11.51 (s, 1H), 9.29 (q, J=1.4 Hz, 1H), 9.00 (s, 1H), 7.99-7.98 (m, 2H), 7.18 (s, 1H); MS ESI+: 448.9 [M+H]⁺.

Charge DMA/CPME=⅓ (5.0 V, 250.0 mL) to the reactor at 25±5° C. under N₂. To a solution of compound D (50.0 g, 1.0 eq) in DMA/CPME::⅓ (250 mL) at 25° C. was added compound E (50.9 g, 1.7 eq) and Cs₂CO₃ (34.4 g, 0.95 eq). The reaction mixture was heated to 80° C. and stirred for 15 h. The mixture was cooled to 25° C. and stirred for 10 h. The mixture was filtered and the filter cake was washed with CPME (2×100 mL). The wet cake was slurried with H₂O (500 mL) and stirred for 3 h. The mixture was filtered and the filter cake was washed with H₂O (2×100 mL). The filter cake was dried for 24 h at 60° C. to afford Compound 1 (50.2 g, 82% yield). ¹H NMR (300 MHz, DMSO) δ 9.32 (t, J=1.5 Hz, 1H), 9.05 (s, 1H), 8.10 (s, 1H), 8.04 (d, J=1.6 Hz, 1H), 7.71 (s, 1H), 7.50-7.43 (m, 1H), 5.15 (t, J=3.8 Hz, 1H), 3.56-3.47 (m, 1H), 3.40-3.35 (m, 1H), 2.41-2.20 (m, 2H), 2.15-2.05 (m, 2H); MS ESI+: 546.0 [M+H]⁺.

### Example 2: Synthesis of (R)-5-(2,5-dichloro-4-(5-(8-chloro-6-(trifluoromethyl)imidazo[1,2-a]pyridin-2-yl)-1,2,4-oxadiazol-3-yl)phenoxy)piperidin-2-one (Compound 1) (alternative synthesis)

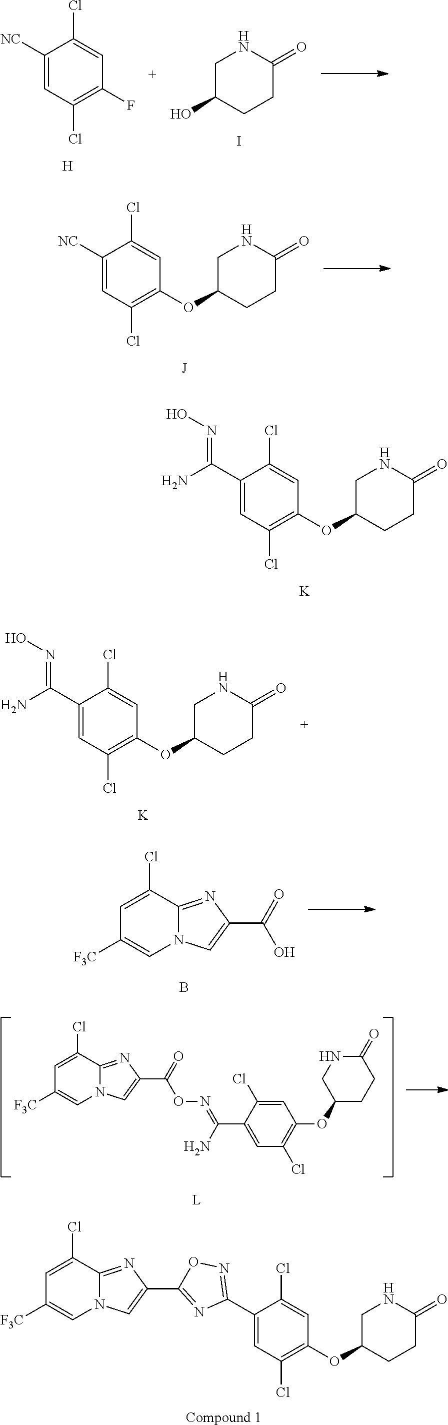

To a solution of compound H (1.0 eq., 50.0 g) in 2-butanone (500 mL) at 25° C. was added K₂CO₃ (55.1 g, 1.5 eq) and compoundI (1.3 eq., 93.1 g). The reaction mixture was heated to 80° C. and stirred for 24 h. The mixture was then cooled to 25° C. Water (500 mL) was added t and the mixture was stirred for 1 h. The mixture was extracted with DCM (4×250 mL). The combined organic phase was washed with water (250 mL) and partially concentrated. MTBE (300 mL) was added and the mixture was stirred 40° C. for 1 h at and then at 25° C. for 1 h. The mixture was filtered and the filter cake was washed with MTBE (2×100 mL). The filter cake was dried for 16 h at 60° C. to afford compound J (36.0 g, 45%). ¹H NMR (300 MHUz, DMSO) δ 8.19 (s, 1H), 7.76 (s, 1H), 7.45 (d, J=2.8 Hz, 1H), 5.14 (p, J=3.7 Hz, 1H), 3.53-3.44 (m, 1H), 3.32-3.29 (m, 1H), 2.33-2.20 (m, 2H), 2.11-2.02 (in, 2H); MS ESI+: 285.0 [M+H]⁺.

To a solution of compound J (25.0 g, 1.0 eq) in DMSO (125 mL) at 25° C. was added NH₂OH (50% w/w in H₂O, 14.5 g, 2.5 eq). The mixture was heated to 50° C. and stirred for 20 h. The mixture was cooled to 0° C. and water (250 mL) and NaCl (100 g) was added. The mixture was stirred at 0° C. for 1 h. The mixture was filtered and the filter cake was washed with water (2×50 mL). The filter cake was dried for 16 h at 60° C. to afford to afford compound K (21.6 g, 76%). ¹H NMR (400 MHUz, DMSO) δ 9.51 (s, 1H), 7.47-7.42 (m, 3H), 5.83 (s, 2H), 5.01 (p, J=3.7 Hz, 1H), 3.45 (ddd, J=13.5, 3.5, 1.4 Hz, 1H), 3.33-3.27 (m, 1H), 2.36-2.17 (m, 2H), 2.09-1.98 (m, 2H); MS ESI+: 318.0 [M+H]⁺.

To a solution of compound B (4.4 g, 1.0 eq)) in DCM (50.0 mL) at 25±5° C. was added HATU (6.6 g, 1.1 eq) and compound K (1.0 eq., 5.0 g). The mixture was stirred for 0.5 h. DIEA (4.1 g, 1.1 eq) was added and the reaction mixture was stirred for 5 h. DMSO (30 mL) was added and the mixture was partially concentrated. Water (100 mL) was added and the mixture was stirred stir for 1 h. The mixture was filtered and washed with H₂O (2×10.0 mL). The filter cake was dried for 18 h at 60° C. to afford compound L (8.3 g, 90% yield). ¹H NMR (400 MHz, DMSO) δ 9.32 (s, 1H), 9.05 (s, 1H), 8.09 (s, 1H), 8.03 (s, 1H), 7.71 (s, 1H), 7.46 (s, 1H), 5.15 (s, 1H), 3.52 (d, J=13.5 Hz, 1H), 3.38 (d, J=13.3 Hz, 1H), 2.36-2.24 (m, 2H), 2.13-2.07 (m, 2H); MS ESI+: 564.0 [M+H]⁺.

To a solution of compound L (5.0 g, 1.0 eq) in DCM (30 mL) at 25° C. was added DBU (2.2 g, 2.0 eq). The reaction mixture was heated to 40° C. and stirred for 6 h. DMAc (100 mL) was added and the mixture was partially concentrated at 45° C. Water (225.0 mL) was added dropwise and the mixture was stirred for 1 h. The mixture was filtered and the filter cake was washed with H₂O (2×10 mL). The filter cake was dried for 18 h at 60° C. to afford Compound 1 (4.3 g, 87%). ¹HNMR (300 MHz, DMSO): 9.32 (t, J=1.5 Hz, 1H), 9.05 (s, 1H), 8.10 (s, 1H), 8.04 (d, J=1.6 Hz, 1H), 7.71 (s, 1H), 7.50-7.43 (m, 1H), 5.15 (t, J=3.8 Hz, 1H), 3.56-3.47 (m, 1H), 3.40-3.35 (m, 1H), 2.41-2.20 (m, 2H), 2.15-2.05 (m, 2H); MS ESI+: 546.0 [M+H]⁺.

### Example 3: Synthesis of (R)-5-(2,5-dichloro-4-(5-(8-chloro-6-(trifluoromethyl)imidazo[1,2-a]pyridin-2-yl)-1,2,4-oxadiazol-3-yl)phenoxy)piperidin-2-one acetic acid salt (Compound 1A)

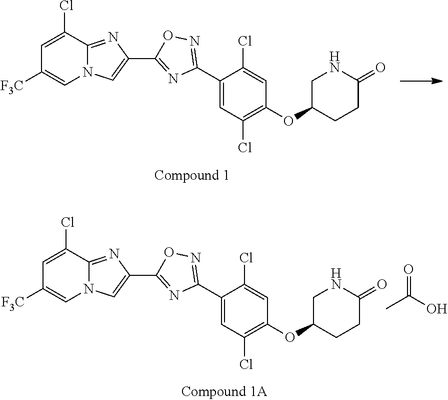

Acetic acid (400 mL) and compound 1 (47.4 g) were heated at 95° C. for 1 h. The mixture was cooled to 55° C. and filtered through activated charcoal. The activated charcoal was washed with acetic acid (2×100 mL) and partially concentrated. Heptane (900 mL) was added and the mixture was stirred for 55° C. for 1 h. The mixture was cooled to 25° C. and stirred for 12 h. The mixture was filtered and the filter cake was washed with heptane (2×250 mL). The filter cake was dried at 25° C. for 1 h to afford compound 1A (39.3 g, 71%). ¹H NMR (300 MHz, DMSO): 11.93 (s, 1H), 9.34-9.29 (m, 1H), 9.05 (s, 1H), 8.10 (s, 1H), 8.04 (d, J=1.6 Hz, 1H), 7.72 (s, 1H), 7.47 (s, 1H), 5.15 (t, J=3.8 Hz, 1H), 3.56-3.48 (m, 1H), 3.40-3.39 (m, 1H), 2.41-2.20 (m, 2H), 2.16-2.05 (m, 2H), 1.91 (s, 3H); MS ESI+. 546.0 [M+H]⁺.

### Example 4: GTPyS Binding Assay

S1P1 membrane is prepared from CHO-K1 Gαqi5 cells expression full-length human S1P1. Scintillation proximity assay (SPA) is performed by incubating membranes, GTPγ³⁵S, and compounds at various concentrations for 60 minutes. Wheat germ agglutinin-coated SPA beads are added and incubated for 60 minutes before centrifugation and scintillation counting. Compound 1 EC50 is less than 1 μM.

The examples and embodiments described herein are for illustrative purposes only and in some embodiments, various modifications or changes are to be included within the purview of disclosure and scope of the appended claims.

What is claimed is:

1. A process for the (R)-5-(2,5-dichloro-4-(5-(8-chloro-6-(trifluoromethyl)imidazo[1,2-a]pyridin-2-yl)-1,2,4-oxadiazol-3-yl)phenoxy)piperidin-2-one (Compound 1), comprising:
A) the reaction of the compounds with the structures: 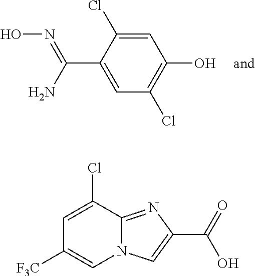 [SMILES: N/C(=N\O)c1cc(Cl)c(O)cc1Cl ; O=C(O)c1cn2cc(C(F)(F)F)cc(Cl)c2n1]
with HOBT and EDCI to produce a compound with the structure: 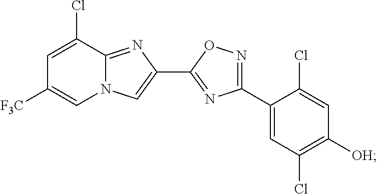 [SMILES: Oc1cc(Cl)c(-c2noc(-c3cn4cc(C(F)(F)F)cc(Cl)c4n3)n2)cc1Cl]
B) followed by the reaction of the compounds with the structures: 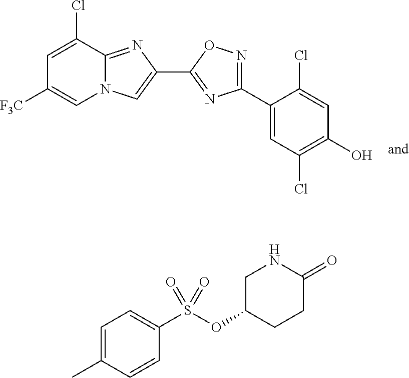 [SMILES: Oc1cc(Cl)c(-c2noc(-c3cn4cc(C(F)(F)F)cc(Cl)c4n3)n2)cc1Cl ; Cc1ccc(S(=O)(=O)O[C@H]2CCC(=O)NC2)cc1]
and With cesium carbonate to produce (R)-5-(2,5-dichloro-4-(5-(8-chloro-6-(trifluoromethyl)imidazo[1,2-a]pyridin-2-yl)-1,2,4-oxadiazol-3-yl)phenoxy)piperidin-2-one (Compound 1) having the structure:

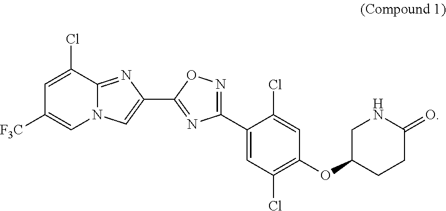 [SMILES: O=C1CC[C@@H](Oc2cc(Cl)c(-c3noc(-c4cn5cc(C(F)(F)F)cc(Cl)c5n4)n3)cc2Cl)CN1]

2. A process for the (R)-5-(2,5-dichloro-4-(5-(8-chloro-6-(trifluoromethyl)imidazo[1,2-a]pyridin-2-yl)-1,2,4-oxadiazol-3-yl)phenoxy)piperidin-2-one (Compound 1), comprising:
A) the reaction of the compounds with the structures: 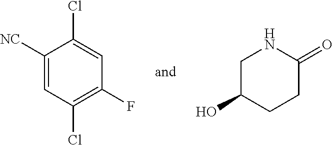 [SMILES: N#Cc1cc(Cl)c(F)cc1Cl ; O=C1CC[C@@H](O)CN1]
with potassium carbonate to produce a compound with the structure: 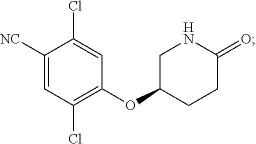 [SMILES: N#Cc1cc(Cl)c(O[C@@H]2CCC(=O)NC2)cc1Cl]
B) followed by the reaction of the compound with the structure: 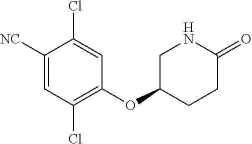 [SMILES: N#Cc1cc(Cl)c(O[C@@H]2CCC(=O)NC2)cc1Cl]
with hydroxylamine to produce a compound with the structure: 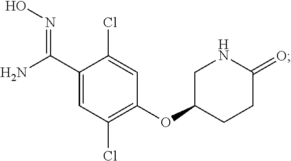 [SMILES: N/C(=N\O)c1cc(Cl)c(O[C@@H]2CCC(=O)NC2)cc1Cl]
C) the reaction of the compounds with the structures: 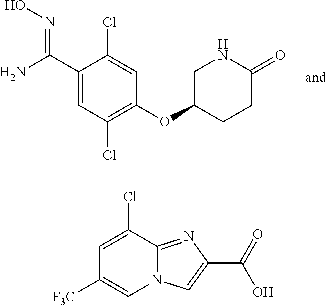
with HATU and DIEA to produce (R)-5-(2,5-dichloro-4-(5-(8-chloro-6-(trifluoromethyl)imidazo[1,2-a]pyridin-2-yl)-1,2,4-oxadiazol-3-yl)phenoxy)piperidin-2-one (Compound 1) having the structure:

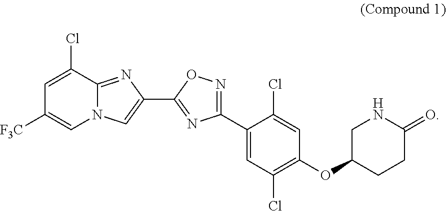 [SMILES: O=C1CC[C@@H](Oc2cc(Cl)c(-c3noc(-c4cn5cc(C(F)(F)F)cc(Cl)c5n4)n3)cc2Cl)CN1]

3. The process of claim 1 or claim 2, further comprising the reaction of (R)-5-(2,5-dichloro-4-(5-(8-chloro-6-(trifluoromethyl)imidazo[1,2-a]pyridin-2-yl)-1,2,4-oxadiazol-3-yl)phenoxy)piperidin-2-one (Compound 1) with acetic acid to produce (R)-5-(2,5-dichloro-4-(5-(8-chloro-6-(trifluoromethyl)imidazo[1,2-a]pyridin-2-yl)-1,2,4-oxadiazol-3-yl)phenoxy)piperidin-2-one acetic acid salt (Compound 1A).

4. A process for the preparation of (R)-5-(2,5-dichloro-4-(5-(8-chloro-6-(trifluoromethyl)imidazo[1,2-a]pyridin-2-yl)-1,2,4-oxadiazol-3-yl)phenoxy)piperidin-2-one (Compound 1): 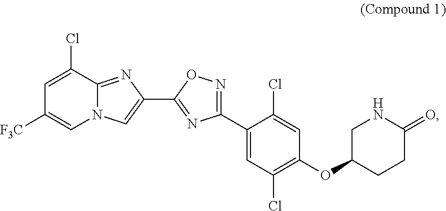 [SMILES: O=C1CC[C@@H](Oc2cc(Cl)c(-c3noc(-c4cn5cc(C(F)(F)F)cc(Cl)c5n4)n3)cc2Cl)CN1]
comprising contacting the compounds with the structures: 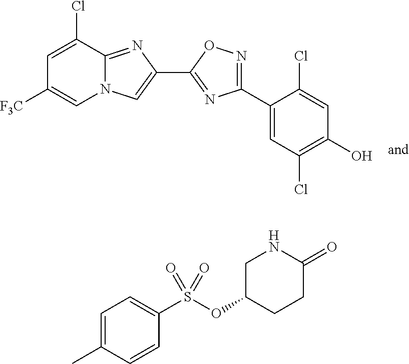 [SMILES: Oc1cc(Cl)c(-c2noc(-c3cn4cc(C(F)(F)F)cc(Cl)c4n3)n2)cc1Cl ; Cc1ccc(S(=O)(=O)O[C@H]2CCC(=O)NC2)cc1]
with a base in the presence of a solvent.

5. The process of claim 4, wherein the base is selected from cesium carbonate, potassium carbonate, potassium phosphate, DBU, sodium t-butoxide, potassium t-butoxide, LiHMDS, NaHMDS, KHMDS, and mixtures thereof.

6. The process of claim 4 or claim 5, wherein the base is cesium carbonate.

7. The process of claim 4 or claim 5, wherein the base is a mixture of cesium carbonate and potassium phosphate.

8. The process of any one of claims 4-7, wherein the solvent is selected from dimethylacetamide, cyclopentyl methyl ether, DMF, NMP, THF, MeTHF, acetonitrile, toluene, and mixtures thereof.

9. The process of any one of claims 4-8, wherein the solvent is a mixture of dimethylacetamide and cyclopentyl methyl ether.

10. The process of any one of claims 4-9, wherein the compound with the structure: 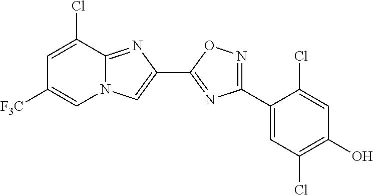 [SMILES: Oc1cc(Cl)c(-c2noc(-c3cn4cc(C(F)(F)F)cc(Cl)c4n3)n2)cc1Cl]
is prepared by a process comprising contacting the compounds with the structures: 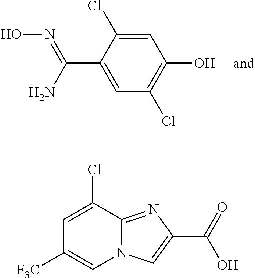 [SMILES: N/C(=N\O)c1cc(Cl)c(O)cc1Cl ; O=C(O)c1cn2cc(C(F)(F)F)cc(Cl)c2n1]
and with a coupling agent in the presence of a solvent.

11. The process of claim 10, wherein the coupling agent is selected from HOBT and EDCI, thionyl chloride, oxalyl chloride, COMU (1-[(1-(cyano-2-ethoxy-2-oxoethylideneaminooxy) dimethylaminomorpholino)]uronium hexafluorophosphate), HBTU (2-(1H-benzotriazole-1-yl)-1,1,3,3-tetramethyluronium hexafluorophosphate), HCTU (O-(1H-6-chlorobenzotriazole-1-yl)-1,1,3,3-tetramethyluronium hexafluorophosphate), TCFH (chloro-N,N,N′,N′-tetramethylformamidinium hexafluorophosphate), phenylboronic acid, silicon tetrachloride, and tetraethyl orthosilicate.

12. The process of claim 10 or claim 11, wherein the coupling agent is HOBT and EDCI.

13. The process of any one of claims 10-12, wherein the solvent is selected from N-methyl-2-pyrrolidone, DMF, DMAc, toluene, MeTHF, and acetonitrile.

14. The process of claim 7 or claim 8, wherein the solvent is N-methyl-2-pyrrolidone.

15. A process for the preparation of (R)-5-(2,5-dichloro-4-(5-(8-chloro-6-(trifluoromethyl)imidazo[1,2-a]pyridin-2-yl)-1,2,4-oxadiazol-3-yl)phenoxy)piperidin-2-one (Compound 1): 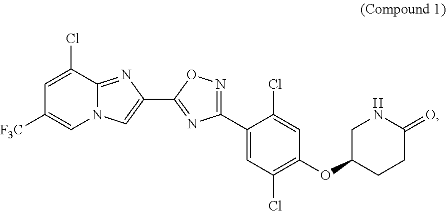 [SMILES: O=C1CC[C@@H](Oc2cc(Cl)c(-c3noc(-c4cn5cc(C(F)(F)F)cc(Cl)c5n4)n3)cc2Cl)CN1]
comprising contacting the compounds with the structures: 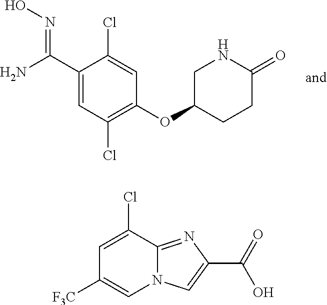
with a coupling agent and a base in the presence of a solvent.

16. The process of claim 15, wherein the coupling agent is selected from HATU, thionyl chloride, oxalyl chloride, COMU (1-[(1-(cyano-2-ethoxy-2-oxoethylideneaminooxy) dimethylaminomorpholino)]uronium hexafluorophosphate), HBTU (2-(1H-benzotriazole-1-yl)-1,1,3,3-tetramethyluronium hexafluorophosphate), HCTU (O-(1H-6-chlorobenzotriazole-1-yl)-1,1,3,3-tetramethyluronium hexafluorophosphate), TCFH (chloro-N,N,N′,N′-tetramethylformamidinium hexafluorophosphate), phenylboronic acid, silicon tetrachloride, and tetraethyl orthosilicate.

17. The process of claim 15 or claim 16, wherein the coupling agent is HATU.

18. The process of any one of claims 15-17, wherein the base is selected from diisopropylethylamine, triethylamine, N-methylmorpholine, N-methylimidazole, and N-methylpiperidine.

19. The process of any one of claims 15-18, wherein the base is diisopropylethylamine.

20. The process of any one of claims 15-19, wherein the solvent is selected from dichloromethane, THF, MeTHF, acetonitrile, NMP, DMAc, DMF, and toluene.

21. The process of any one of claims 15-20, wherein the solvent is dichloromethane.

22. The process of any one of claims 15-21, wherein the compound with the structure: 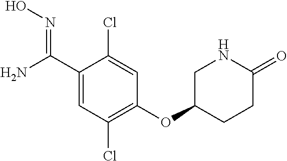 [SMILES: N/C(=N\O)c1cc(Cl)c(O[C@@H]2CCC(=O)NC2)cc1Cl]
is prepared by a process comprising contacting the compound with the structure: 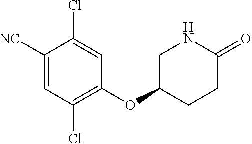 [SMILES: N#Cc1cc(Cl)c(O[C@@H]2CCC(=O)NC2)cc1Cl]
with hydroxylamine in the presence of a solvent.

23. The process of claim 22, wherein the solvent is selected from dimethyl sulfoxide, DMF, DMAc, NMP, MeOH, EtOH, and 2-PrOH.

24. The process of claim 22 or claim 23, wherein the solvent is dimethyl sulfoxide.

25. The process of any one of claims 15-24, wherein the compound with the structure: 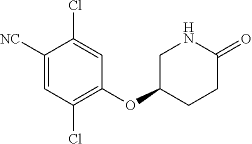 [SMILES: N#Cc1cc(Cl)c(O[C@@H]2CCC(=O)NC2)cc1Cl]
is prepared by a process comprising contacting the compounds with the structures: 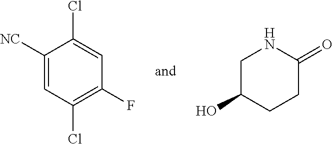 [SMILES: N#Cc1cc(Cl)c(F)cc1Cl ; O=C1CC[C@@H](O)CN1]
with a base in the presence of a solvent.

26. The process of claim 25, wherein the base is selected from potassium carbonate, DBU, sodium t-butoxide, potassium t-butoxide, LiHMDS, NaHMDS, and KHMDS.

27. The process of claim 25 or claim 26, wherein the base is potassium carbonate.

28. The process of any one of claims 25-27, wherein the solvent is selected from 2-butanone, DMF, DMAc, NMP, THF, and acetonitrile.

29. The process of any one of claims 25-28, wherein the solvent is 2-butanone.

30. A compound with the structure:

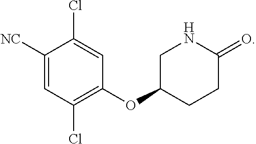 [SMILES: N#Cc1cc(Cl)c(O[C@@H]2CCC(=O)NC2)cc1Cl]

31. A compound with the structure: 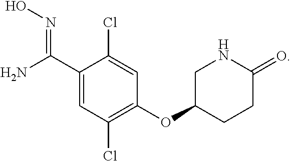 [SMILES: N/C(=N\O)c1cc(Cl)c(O[C@@H]2CCC(=O)NC2)cc1Cl]
